(19) United States
(12) Patent Application Publication
(10) Pub. No.: US 2021/0393656 A1
(43) Pub. Date: Dec. 23, 2021

(54) A PROCESS FOR PREPARATION OF A STABLE PHARMACEUTICAL COMPOSITION OF BORTEZOMIB

(71) Applicant: INTAS PHARMACEUTICALS LTD., Ahmedabad, Gujarat (IN)

(72) Inventors: Kirti MAHESHWARI, Gujarat (IN); Alex GEORGE, Gujarat (IN); Mukesh BOTHRA, Gujarat (IN); Anil NAYANI, Gujarat (IN)

(21) Appl. No.: 17/419,129

(22) PCT Filed: Jan. 9, 2020
(86) PCT No.: PCT/IB2020/050136

§ 371 Date: Jun. 28, 2021

(30) Foreign Application Priority Data
Jan. 11, 2019 (IN) 201921001429

(51) Int. Cl. A61K 31/69; A61K 47/26; A61K 9/08

(52) U.S. Cl. CPC A61K 31/69; A61K 9/08; A61K 47/26

(57) ABSTRACT

The present invention relates to a process for preparation of a stable pharmaceutical composition of Bortezomib. The said pharmaceutical composition of Bortezomib provides an improved stability compared to the reconstituted solution of the lyophilized product.

### RELATED APPLICATIONS

This application is related to Indian Provisional Application No. IN201921001429 filed on 11^{th} Jan. 2019 and is incorporated herein in its entirety.

### FIELD OF THE INVENTION

The present invention relates to a process for preparation of a stable ready-to-use pharmaceutical composition of bortezomib. The said pharmaceutical composition of bortezomib provides an improved stability as compared to the reconstituted solution of the lyophilized product.

### BACKGROUND OF THE INVENTION

Bortezomib, chemically known as [(1R)-3-methyl-1[[(2S)-1-oxo-3-phenyl-2-[(pyrazinylcarbonyl) amino]propyl]amino]butyl] boronic acid, is a proteasome inhibitor approved for use in treating various neoplastic diseases, and especially treatment of relapsed multiple myeloma and mantle cell lymphoma.

Bortezomib is susceptible to degradation in the aqueous solutions, and hence it is available as a lyophilized powder namely VELCADE®. The commercially available lyophilized powder contains 3.5 mg of bortezomib and 35 mg of mannitol in a single-dose vial. However, this lyophilized product requires the reconstitution step prior to use, and the reconstituted solution should be administered within 8 hours of preparation. The reconstituted product is having limited stability, in particular when bortezomib is desired to be given as a storage-stable pharmaceutical composition.

The U.S. patents numbered U.S. Pat. Nos. 6,713,446 and 6,958,319 describes a stable pharmaceutical composition of boronic acid compounds, in particular bortezomib-mannitol ester. The U.S. patents numbered U.S. Pat. Nos. 8,263,578 and 9,061,037 describe a non-aqueous pharmaceutical composition of bortezomib, wherein the solvent system is substantially non-aqueous system comprising propylene glycol as a predominant component. The U.S. Patent Application No. 20170143622 describes a liquid pharmaceutical composition of bortezomib, wherein the solvent system comprises less than about 50% v/v of non-aqueous solvent. The PCT patent applications numbered WO2017013208 and WO2017013209 describe a ready-to-use solution of bortezomib, wherein the mannitol is dissolved in water with stirring and nitrogen gas was passed to reduce oxygen content. Further, mannitol solution was heated at temperature of 45° C., and bortezomib was subsequently dissolved in the heated mannitol solution with stirring. Then the bortezomib-mannitol solution was sterile-filtered and filled under nitrogen atmosphere into vials. The filled vials are then transferred to the lyophilization chamber and sealed under nitrogen atmosphere. Since Bortezomib is a low water-soluble drug, the prior art process involved in the PCT applications may be more time-consuming and challenging.

Although, the prior art approach discloses bortezomib compositions in the form of lyophilized powder and non-aqueous solution, it has been very challenging to produce a ready-to-use solution of bortezomib, which can provide improved solubility and long-term stability. Moreover, the processes known in the art are complicated, time-consuming and costly. Therefore, there exists a need for a process for preparation of a stable pharmaceutical composition of bortezomib that overcomes all the issues as mentioned above.

The inventors of present invention have developed an advantageous process for preparation of a stable ready-to-use pharmaceutical composition of bortezomib.

### OBJECTS OF THE INVENTION

The main objective of the present invention is to provide a process for preparation of a stable ready-to-use pharmaceutical composition of bortezomib, which provides an improved stability as compared to the reconstituted solution of the lyophilized product.

Another object of the present invention is to provide a process for the preparation of a stable ready-to-use pharmaceutical composition of bortezomib, wherein the said process does not require a lyophilization step.

Another object of the present invention is to provide a process for the preparation of a stable ready-to-use pharmaceutical composition of bortezomib, wherein the said process involves the use of vacuum evaporation to obtain a substantially aqueous composition of bortezomib.

### SUMMARY OF THE INVENTION

In a first embodiment, the present invention relates to a process for the preparation of a stable ready-to-use pharmaceutical composition of bortezomib, wherein the said process does not require a lyophilization step.

In another embodiment, the present invention relates to a process for the preparation of a stable ready-to-use pharmaceutical composition of bortezomib, wherein the said process involves the use of vacuum evaporation to obtain a substantially aqueous composition of bortezomib.

In another embodiment, the present invention relates to a process for preparation of a stable ready-to-use pharmaceutical composition; wherein the process comprises steps of:
a) Preparing a bulk solution comprising bortezomib, mannitol, non-aqueous solvent and water for injection, with inert gas sparging into bulk solution to achieve dissolved oxygen content below 1.5 ppm,
b) Removing the non-aqueous solvent from bulk solution by vacuum evaporation at specific temperature, rotation speed and vacuum, to obtain substantially aqueous solution after vacuum evaporation,
c) Adding mannitol and water for injection into the obtained solution to make up isotonicity and desired bortezomib concentration in the solution,
d) Filter-sterilizing the obtained solution and filling the final solution into a suitable container.

### DETAILED DESCRIPTION

The detailed description and the examples provided herein are exemplary and any modification or variation within the scope of the invention will be apparent to a person skilled in the art. Further, unless otherwise defined, all the technical and scientific terms used herein shall bear the meaning as understood by a person who is ordinarily skilled in the art.

In one aspect, the present invention provides a stable ready-to-use pharmaceutical composition comprising bortezomib, mannitol and water for injection.

In another aspect, the present invention relates to a process for the preparation of a stable ready-to-use pharmaceutical composition of bortezomib, wherein the said process involves the use of vacuum evaporation to obtain a substantially aqueous solution of bortezomib.

In another embodiment, the pharmaceutical composition comprises from about 0.01 mg/ml to about 25 mg/ml of bortezomib or pharmaceutically acceptable salts thereof. In a preferred embodiment, the said composition is isotonic and comprises 2.5 mg/ml of bortezomib or pharmaceutically acceptable salts and esters thereof. Further, the “Bortezomib” drug of the present invention includes bortezomib in a free form, bortezomib-mannitol ester, bortezomib-boric acid, and other pharmaceutically acceptable salts or esters thereof.

The stable pharmaceutical composition of the present invention has sufficient stability to allow storage at a convenient temperature, preferably between −20° C. and 25° C., more preferably about 2° C. to about 8° C., for a reasonable period. The term “stable” used in the present invention means that the assay of bortezomib in the said composition is not less than 90% during the shelf life of the composition. The assay of Bortezomib in the pharmaceutical composition can be carried out by any of the methods known to a person skilled in the art. In a preferred embodiment, the assay is performed by HPLC method.

The shelf-life of the said composition can be as short as three months, but is typically six months or longer, more preferably one year or two years. In another embodiment, the ready-to-use composition comprises 2.5 mg/ml of bortezomib, wherein the assay of bortezomib is not less than 90% (i.e. NLT 90%). The pharmaceutical composition according to the present invention is stored at 2° C. to 8° C. throughout the shelf life.

The impurities related to bortezomib composition are characterized as follows: Specified Impurity-1: (S)-3-phenyl-2-(pyrazine-2-carboxamido) propanoic acid; Specified Impurity-2: (N-(1-[((R)-1-Hydroxy-3-Methylbutyl)Amino)-1-oxo-3-Phenylpropan-2yl] Pyrazine-2-carboxamide);

Specified Impurity-3: N-(1-amino-1-oxo-3-phenylpropan-2-yl)pyrazine-2-carboxamide.

In a preferred embodiment, the limit for each of the specified impurity-1, specified impurity-2, and specified impurity-3 in the stable ready-to-use pharmaceutical composition of bortezomib is not more than (NMT) 1.0%.

In a preferred embodiment, the limit for single unknown impurity in the stable ready-to-use pharmaceutical composition of bortezomib is not more than 0.5%.

In a preferred embodiment, the total impurity in the stable ready-to-use pharmaceutical composition of bortezomib is not more than 3%.

The lyophilization method is complicated and costly. Further, the lyophilized powder requires reconstitution step prior to use, and the reconstituted solution provides very limited stability of about 8 hours. Therefore, the inventors have successfully developed a process for preparation of stable ready-to-use pharmaceutical composition comprising bortezomib, wherein the solvent is removed by vacuum evaporation process using a rotary vacuum evaporator. Such compositions provide an improved stability as compared to the reconstituted solution of the lyophilized product.

The term “Vacuum Evaporation” is referred to as a process for solvent removal by controlling the temperature and vacuum pressure in a suitable equipment such as rotary vacuum evaporator. The process of vacuum evaporation is performed at specific temperature, time, rotation speed and vacuum. In a preferred embodiment, the heating temperature is from about 30-60° C., more preferably at 50° C.; the rotation speed is about 30-100, more preferably at 50 RPM; the vacuum is about 30-120 mbar; and the time-period for the process depends on batch size and capacity. In another preferred embodiment, the solvent removal process in vacuum evaporator involves the steps of setting the chiller temperature (−5° C.) for condensation; and setting the heating bath temperature to 50° C. for heating the solution in the rotary evaporator. Furthermore, to prevent foaming in evaporating flask, the vacuum is adjusted gradually to 700 mbar at 20 RPM and then reduced to 30 mbar slowly with increasing rotation speed up to 65 RPM. The inert gas sparging is carried out in order to prevent oxidation degradation of Bortezomib. In a preferred embodiment, the inert gas is nitrogen for sparging into bulk solution to achieve dissolved oxygen content below 1.5 ppm.

Further, the compositions of the present invention may comprise other pharmaceutically acceptable excipients selected from solvents, bulking agents, complexing agents, preservatives, anti-oxidants, stabilizers, tonicity modifiers or any other suitable excipients thereof.

The solvents, for the purpose of the present invention, include alcohols, glycols, polar protic and aprotic solvents. In a preferred embodiment, the solvent is tertiary butanol and ethanol. In the most preferred embodiment, the solvent is tertiary butanol (TBA). The bulk solution used in the present invention comprises a non-aqueous solvent and water for injection. During the process for preparation of the final solution, the non-aqueous solvent is removed from the bulk solution by vacuum evaporation, and the obtained solution is substantially aqueous solution that may contain negligible amount of non-aqueous solvent. In a preferred embodiment, the content of tertiary butanol is not more than 5000 ppm (i.e. NMT 5000 ppm).

The bulking agents for the purpose of the present invention include saccharides, preferably monosaccharides or oligosaccharides, sugar alcohols, and other suitable excipients thereof. The suitable bulking agents include the following, but are not limited to mannitol, sodium chloride, glucose, sucrose, lactose, trehalose, dextrose, maltose, sorbitol, dextran, povidone, amino acids such as glycine, arginine, aspartic acid and mixtures thereof. In a preferred embodiment, the bulking agent is mannitol. Further, the composition of the present invention may be diluted with an intravenous admixture, such as normal saline.

The stable bortezomib composition of the present invention can be packaged in a suitable container such as pre-filled syringe (PFS), vial, ampoule, and infusion bag. In a preferred embodiment, the container for packaging is PFS pack or vial pack. The pH of the composition of the present invention is between 4.0 to 7.0.

The stable bortezomib composition of the present invention can be used in the treatment of diseases such as multiple myeloma, mantle cell lymphoma, leukemia's, cancer and autoimmune diseases.

In order to further illustrate the present invention, the following examples are provided for the purpose of clarity of understanding. However, it is not intended in any way to limit the scope of present invention and it is readily apparent to those of ordinary skill in the art in light of the teachings of this invention that certain changes and modifications may be made thereto without departing from the scope of the invention.

### EXAMPLE 1

### Stable Pharmaceutical Composition of Bortezomib

Bortezomib Injection

| Sr. No. | Ingredient | Quantity (mg/ml) |
|---|---|---|
| 1. | Bortezomib | 0.01-25 mg/ml |
| 2. | Mannitol | q.s. |
| 3. | Non-aqueous Solvent | q.s |
| 4. | Water for Injection | q.s. to 1 ml |
| 5. | Nitrogen | q.s. to sparge |

### Manufacturing Process

The bulk solution for making a stable composition of bortezomib can be prepared by following steps:
a) Preparing a bulk solution comprising bortezomib, mannitol, non-aqueous solvent and water for injection, with nitrogen gas sparging into bulk solution to achieve dissolved oxygen content below 1.5 ppm,
b) Removing the non-aqueous solvent from bulk solution by vacuum evaporation at specific temperature, rotation speed and vacuum, and obtaining the substantially aqueous solution after vacuum evaporation,
c) Adding mannitol and water for injection into the obtained solution to make up isotonicity and desired bortezomib concentration in the solution,
d) Filter-sterilizing the obtained solution and filling the final solution into a suitable container.

During the preparation of the composition according to the present invention, the bortezomib-mannitol ester may be formed in the solution.

### EXAMPLE 2

### Stable Pharmaceutical Composition of Bortezomib

Bortezomib Injection

| Sr. No. | Ingredient | Quantity (mg/ml) |
|---|---|---|
| 1. | Bortezomib | 0.01-25 mg/ml |
| 2. | Mannitol | q.s. |
| 3. | Tertiary butanol (TBA) | q.s |
| 4. | Water for Injection | q.s. to 1 ml |
| 5. | Nitrogen | q.s. to sparge |

### Manufacturing Process

The stable composition of bortezomib can be prepared by following steps:
a) Preparing a bulk solution comprising bortezomib, mannitol, tertiary butanol and water for injection, with nitrogen gas sparging into bulk solution to achieve dissolved oxygen content below 1.5 ppm,
b) Removing the tertiary butanol from bulk solution by vacuum evaporation at specific temperature, rotation speed and vacuum, and obtaining the substantially aqueous solution after vacuum evaporation,
c) Adding mannitol and water for injection into the obtained solution to make up isotonicity and desired bortezomib concentration in the solution,
d) Filter-sterilizing the obtained solution and filling the final solution into a suitable container.

### EXAMPLE 3

### Stable Ready-To-Use Pharmaceutical Composition of Bortezomib Stage-1: Bulk Solution Preparation for Bortezomib Composition

Bortezomib Injection 5 mg/ml

| Sr. No. | Ingredient | Quantity (mg/ml) |
|---|---|---|
| 1. | Bortezomib | 5.00 mg/ml^{$} |
| 2. | Mannitol | 50.00 mg/ml |
| 3. | Tertiary Butanol (TBA) | q.s. to 1 ml (320.00 mg/ml) |
| 4. | Water for Injection | 598.64 mg/ml |
| 5. | Nitrogen | q.s. to sparge |

^{$}Mentioned quantity is considering Assay as 100%,
Potency correction to be done while dispensing considering the actual assay on as such basis.

### Manufacturing Procedure for Stage—1

1) Take required amount water for injection into the manufacturing vessel and sparge nitrogen to achieve dissolved oxygen content below 1.5 ppm,
2) Add and dissolve mannitol into water for injection with nitrogen sparging,
3) Add and mix tertiary butanol into solution of step 2 with nitrogen sparging,
4) Prepare a slurry of bortezomib and transfer to manufacturing vessel by rinsing the slurry container with bulk solution of step 3 and stir to get clear solution with nitrogen sparging.

### Manufacturing Stage—II: Solvent Removal by Vacuum Evaporation in Rotary Vacuum Evaporator

5) Take the bulk solution of stage-I into rotary vacuum evaporator,
6) Remove tertiary butanol from the bulk solution by vacuum evaporation in rotary vacuum evaporator. This above step involves the steps of setting the chiller temperature (−5° C.) for condensation, and setting the heating bath temperature to 50° C. for heating the solution in the rotary evaporator.
7) To prevent foaming in evaporator, vacuum is adjusted gradually to 700 mbar at 20 RPM and then reduced to 30 mbar slowly with increasing rotation speed up to 65 RPM.
8) Collect the obtained solution left in evaporating flask after removal of tertiary butanol.

### Manufacturing Stage—III: Final Solution Manufacturing Formula/Composition

Bortezomib Injection 2.5 mg/ml

| Sr. | | Quantity |
|---|---|---|
| No. | Ingredient | (mg/ml) |
| 1. | Bortezomib as mannitol ester | 2.5 mg/ml |
| | (solution from stage-II) | |
| 2. | Mannitol | 25.00 mg/ml |
| 3. | Water for Injection | q.s. to 1 ml |
| 4. | Nitrogen | q.s. to sparge |

### Brief Manufacturing Procedure

9) Transfer the obtained solution after vacuum evaporation into the manufacturing vessel,
10) Add and dissolve mannitol in above solution with nitrogen sparging,
11) Make up the volume with water for injection to yield bortezomib injection as 2.5 mg/ml,
12) Sparge nitrogen gas into obtained solution with stirring to achieve dissolved oxygen content below 1.5 ppm,
13) Filter-sterilize the obtained solution and fill the final solution into a suitable container.

The assay of bortezomib in the said composition of the present invention is not less than 90% during the shelf-life of the composition.

### EXAMPLE 4

### Stability Results for the Ready-to-Use Composition of Bortezomib PFS Pack

The composition of bortezomib as described in the Example-3 was subject to stability studies for 1 month, 2 months, 3 months and 6 months duration at the storage conditions of 25° C. and 2-8° C. respectively, and the results are provided in the table below.

Bortezomib Injection 2.5 mg/ml-Development batches Stability Data-PFS Pack

| | | | TBA | Specified | Specified | Specified | Single | | |
|---|---|---|---|---|---|---|---|---|---|
| | | | content | Impurity- | Impurity- | Impurity- | unknown | Total | |
| | Condi- | pH | NMT | 1 | 2 | 3 | impurity | Impurity | Assay |
| | tions | Between | 5000 | NMT | NMT | NMT | NMT | NMT | NLT |
| Batch No | Limits | 4.0 to 7.0 | ppm | 1.0% | 1.0% | 1.0% | 0.5% | 3.0% | 90% |
| 0.8 ml PFS | Initial | 5.05 | 30.66 | 0.023 | 0.116 | 0.075 | 0.072 | 0.320 | 97.7% |
| pack- | | | | | | | (RRT 1.83) | | |
| Each PFS | 3M, | 4.43 | NA | 0.860 | 0.186 | 0.109 | 0.299 | 1.487 | 95.8% |
| contains: | 25° C. | | | | | | (RRT 1.83) | | |
| Bortezomib: | 3M, | 4.79 | NA | 0.090 | 0.099 | 0.077 | 0.135 | 0.401 | 97.1% |
| 2 mg | 2-8° C. | | | | | | (RRT 1.83) | | |
| | 6M, | 4.83 | NA | 0.137 | 0.094 | 0.073 | 0.143 | 0.486 | 96.7% |
| | 2-8° C. | | | | | | (RRT 1.86) | | |
| | 6M, | 4.78 | NA | 1.631 | 0.312 | 0.162 | 0.519 | 2.802 | 95.4% |
| | 25° C. | | | | | | (RRT 1.86) | | |
| 1.1 ml PFS | Initial | 5.00 | 30.66 | 0.023 | 0.117 | 0.075 | 0.070 | 0.319 | 97.3% |
| pack- | | | | | | | (RRT 1.83) | | |
| Each PFS | 3M, | 4.68 | NA | 0.867 | 0.190 | 0.109 | 0.308 | 1.535 | 95.2% |
| contains: | 25° C. | | | | | | (RRT 1.83) | | |
| Bortezomib: | 3M, | 473 | NA | 0.091 | 0.088 | 0.077 | 0.128 | 0.384 | 97.1% |
| 2.75 mg | 2-8° C. | | | | | | (RRT 1.83) | | |
| | 6M, | 4.76 | NA | 0.136 | 0.088 | 0.072 | 0.135 | 0.477 | 96.6% |
| | 2-8° C. | | | | | | (RRT 1.86) | | |
| | 6M, | 4.68 | NA | 1.637 | 0.278 | 0.151 | 0.473 | 2.718 | 95.7% |
| | 25° C. | | | | | | (RRT 1.86) | | |

RRT = Relative Retention Time.

The results indicate that the pharmaceutical composition of bortezomib as 2.5 mg/ml in PFS pack remains stable, when stored at 25° C. and 2-8° C. for at least three months.

### EXAMPLE 5

### Stability Results for the Ready-to-Use Composition of Bortezomib Vial Pack

The composition of bortezomib as described in the Example-3 was subject to stability studies for 1 month, 2 months, 3 months and 6 months duration at the storage conditions of 25° C. and 2-8° C. respectively, and the results are provided in the table below.

Bortezomib Injection 2.5 mg/ml-Development batches Stability Data-Vial Pack

| | | | TBA | Specified | Specified | Specified | Single | | |
|---|---|---|---|---|---|---|---|---|---|
| | | | content | Impurity- | Impurity- | Impurity- | unknown | Total | |
| | Condi- | pH | NMT | 1 | 2 | 3 | impurity | Impurity | Assay |
| | tions | Between | 5000 | NMT | NMT | NMT | NMT | NMT | NLT |
| Batch No | Limits | 4.0 to 7.0 | ppm | 1.0% | 1.0% | 1.0% | 0.5% | 3.0% | 90% |
| 1 ml vial | Initial | 5.10 | 30.66 | 0.023 | 0.118 | 0.075 | 0.072 | 0.322 | 97.7% |
| pack | | | | | | | (RRT 1.84) | | |
| Each Vial | 3M, | 5.24 | NA | 0.936 | 0.127 | 0.123 | 0.188 | 1.483 | 96.2% |
| contains: | 25° C. | | | | | | (RRT 1.83) | | |
| Bortezomib: | 3M, | 5.24 | NA | 0.094 | 0.091 | 0.080 | 0.115 | 0.380 | 97.5% |
| 2.5 mg | 2-8° C. | | | | | | (RRT 1.83) | | |
| | 6M, | 5.25 | NA | 1.739 | 0.178 | 0.167 | 0.285 | 2.616 | 94.9% |
| | 25° C. | | | | | | (RRT 1.86) | | |
| | 6M, | 5.31 | NA | 0.143 | 0.085 | 0.076 | 0.123 | 0.478 | 98.1% |
| | 2-8° C. | | | | | | (RRT 1.86) | | |
| 1.4 ml vial | Initial | 5.20 | 210 | 0.038 | 0.115 | 0.090 | 0.085 | 0.360 | 98.1% |
| pack | | | | | | | (RRT 1.84) | | |
| Each Vial | 3M, | 5.06 | NA | 0.968 | 0.142 | 0.126 | 0.190 | 1.598 | 95.2% |
| contains: | 25° C. | | | | | | (RRT 1.87) | | |
| Bortezomib: | 3M, | 5.09 | NA | 0.086 | 0.109 | 0.081 | 0.110 | 0.442 | 96.4% |
| 3.5 mg | 2-8° C. | | | | | | (RRT 1.86) | | |
| | 6M, | 5.01 | NA | 1.897 | 0.140 | 0.174 | 0.228 | 2.668 | 93.8% |
| | 25° C. | | | | | | (RRT 1.84) | | |
| | 6M, | 5.08 | NA | 0.163 | 0.081 | 0.080 | 0.122 | 0.446 | 97.5% |
| | 2-8° C. | | | | | | (RRT 1.84) | | |

The results indicate that the pharmaceutical composition of bortezomib as 2.5 mg/ml in vial pack remains stable, when stored at 25° C. and 2-8° C. for at least three months.

The stability data for the pharmaceutical compositions obtained by the process of the present invention stored at 2° C. to 8° C. is the real-time stability data, whereas the stability data for pharmaceutical compositions stored at 25° C. is the accelerated stability data. The said pharmaceutical composition of the present invention is stored at 2° C. to 8° C. through-out the shelf life.

We claim:

1. A stable ready-to-use pharmaceutical composition of bortezomib obtained by a process, wherein the process comprises use of vacuum evaporation to obtain a substantially aqueous composition of bortezomib.

2. The stable ready-to-use pharmaceutical composition of bortezomib obtained by a process according to claim 1, wherein the process does not require a lyophilization step.

3. The stable ready-to-use pharmaceutical composition of bortezomib according to claim 1, wherein the amount of bortezomib or pharmaceutically acceptable salts thereof is from about 0.01 mg/ml to about 25 mg/ml.

4. The stable ready-to-use pharmaceutical composition of bortezomib according to claim 1, wherein the composition comprises bortezomib, mannitol and water for injection.

5. A process for preparation of a stable ready-to-use pharmaceutical composition of 0.01 mg/ml to about 25 mg/ml of bortezomib comprising the steps of:
i) Preparing a bulk solution comprising bortezomib, mannitol, non-aqueous solvent and water for injection, with sparging of inert gas into bulk solution to achieve dissolved oxygen content below 1.5 ppm,
ii) Removing the non-aqueous solvent from bulk solution by vacuum evaporation with heating temperature from about 30-60° C., rotation speed from about 30-100 RPM, and vacuum from about 30-120 mbar to obtain the substantially aqueous solution after vacuum evaporation process,
iii) Adding mannitol and water into the obtained solution to make the final solution comprising from about 0.01 mg/ml to about 25 mg/ml of bortezomib,
iv) Filter-sterilizing and filling the bortezomib solution into a suitable container.

6. The stable ready-to-use pharmaceutical composition of bortezomib obtained by the process according to claim 5, wherein the total specified impurity in the composition is not more than 3%, when stored at a temperature of 2-8° C. and 25° C. for at least three months.

7. The stable ready-to-use pharmaceutical composition of bortezomib obtained by the process according to claim 5, wherein the unknown impurity in the composition is not more than 0.5%, when stored at a temperature of 2-8° C. and 25° C. for at least three months.

8. The stable ready-to-use pharmaceutical composition of bortezomib obtained by the process according to claim 5, wherein the composition is filled into a pre-filled syringe or a vial.

9. The stable ready-to-use pharmaceutical composition of bortezomib obtained by the process according to claim 5, wherein the pH of the composition is between 4.0 to 7.0.

10. The stable ready-to-use pharmaceutical composition of bortezomib obtained by the process according to claim 5, wherein composition can be used for the treatment of multiple myeloma and mantle cell lymphoma.
